(19) United States
(12) Patent Application Publication
(10) Pub. No.: US 2003/0171662 A1
(43) Pub. Date: Sep. 11, 2003

(54) Non-adhesive flexible electro-optical sensor for fingertip trans-illumination

(76) Inventors: Michael William O'Connor, Frisco, TX (US); Tracie Lynn Patton, Plano, TX (US); Jeffrey Albert Secunda, Dallas, TX (US)

Correspondence Address: BRACEWELL & PATTERSON, L.L.P., Intellectual Property Law, P.O. Box 969, Austin, TX, 78767-0969, US

(21) Appl. No.: 10/092,827

(22) Filed: Mar. 7, 2002

(51) Int. Cl. A61B 5/00

(52) U.S. Cl. 600/323

(57) ABSTRACT

A sensor for trans-illumination of a blood perfused portion of a patient. The sensor includes a flexible, initially substantially planar web-like support structure which has an adhesive layer on an upper surface thereof and a loop fabric backing on a lower surface thereof, a light source and a photo-sensor are mounted to the adhesive layer and a compressible foam layer having apertures which overlie the light source and the photo-sensor is mounted to the adhesive layer. A hook fabric tab is mounted to one end of the web-like support structure so that the web-like support structure maybe wrapped around and secured to a patient's finger, toe, hand or foot.

### BACKGROUND OF THE INVENTION

1. Technical Field

The present invention relates, in general, to improved electro-optical sensors for measurement of arterial oxygen saturation and, in particular, to electro-optical sensors for measurement of arterial oxygen saturation which have cutaneous performance. Still more particularly, the present invention relates to an electro-optical sensor for the measurement of arterial oxygen saturation which is mounted within a non-adhesive flexible web-like structure which may be removed and replaced without skin trauma or irritation.

2. Description of the Related Art

Pulse oximetry involves the continuous, non-invasive monitoring of the oxygen saturation level in blood perfused tissue to provide an early indication of impending shock. An oximeter probe typically is secured to the patient and provides an electrical signal to an oximeter device. The oximeter device houses electronic circuitry for processing this electric signal and generating human-readable indicia of the patient's blood oxygen saturation level. Both disposable and non-disposable sensor probes for this purpose are widely utilized.

Non-disposable probes are typically designed utilizing a clamp design. This design includes one or more light-emitting diodes which are adjacent to one side of a fleshy human appendage, such as a finger. Light from the light-emitting diode is received by a photosensor which is placed on the opposing fleshy side of the appendage. Such devices generally consist of a small spring-loaded clip which attaches like a common clothespin to the tip of a finger or similar appendage. While this technique works quite well in many applications, this design suffers from selected defects. For example, inaccurate measurements may result because of so-called “motion artifact” which is created by differential motion between the sensor and the patient's finger, as well as changes in pressure within the tissue. Further, these clamp-type sensors may become removed inadvertently. Additionally, the spring-loaded pressure on a fleshy tissue over a period of time will cause a reduction of blood flow to that tissue. Reduction of blood flow will cause a concomitant loss of pulse amplitude and, thus, a loss of the optical signal to be measured. To minimize this constructed effect of clamp-type attachments, the sensor must be adjusted or repositioned frequently, generally once or twice per hour. These drawbacks result in this type of clamp sensor being unacceptable for long-term, uninterrupted measurement.

Disposable sensor probes also are known in the prior art. U.S. Pat. No. 4,830,014 discloses a sensor probe which comprises a light source and photosensor mounted within the web of an elongated flexible strip. The flexible strip is then wrapped around the human fingertip, toe, hand or infant's foot such that the light source and sensor are positioned in directly overlying relationship. The low mass and aspect ratio of such sensor probes minimize the motion artifact present within larger clamp-type sensors, and the adhesive nature of the elongated strip causes the sensor and light source to conform to the fingertip skin, minimizing the distortion brought about by pressure on fleshy tissue.

While the sensor disclosed within U.S. Pat. No. 4,830,014 provides relief from several of the defects known to exist within non-disposable sensor probes, these so-called “bandaid” sensors include various defects as well.

For example, the adhesive utilized to obtain cutaneous conformance can result in skin irritation from the chemicals within the adhesive or skin trauma from removal of the sensor. Further, as advantageous as flexible band-aid like sensors are such devices are typically single use and if the sensor must be removed from the patient, a new sensor must typically be obtained and mounted to the patient.

In view of the above, it should be apparent that a need exists for a disposable electro-optical sensor which may be utilized to trans-illuminate a human fingertip, toe, hand or infant's foot which possesses a small mass and which will not irritate the patient's skin and which can be easily removed and replaced.

### SUMMARY OF THE INVENTION

It is, therefore, an object of the present invention to provide an improved electro-optical sensor.

It is another object of the present invention to provide an improved electro-optical sensor for non-invasive photo-electric measurement of arterial oxygen saturation.

It is yet another object of the present invention to provide an improved electro-optical sensor for trans-illumination of a human fingertip, toe, hand or infant's foot which conforms to the skin thereof without irritating or traumatizing the skin as a result of adhesive chemicals or friction.

The foregoing objects are achieved as is now described. A sensor is provided for trans-illumination of a blood perfused portion of a patient. The sensor includes a flexible, initially substantially planar web-like support structure which has an adhesive layer on an upper surface thereof and a loop fabric backing on a lower surface thereof. A light source and photo-sensor are mounted to the adhesive layer and a compressible foam layer having apertures which overlie the light source and the photo-sensor is mounted to the adhesive layer. A hook fabric tab is mounted to one end of the web-like support structure so that the web-like support structure may be wrapped around and secured to a patient's finger, toe, hand or foot.

### BRIEF DESCRIPTION OF THE DRAWINGS

The novel features believed characteristic of the invention are set forth in the appended claims. The invention itself, however, as well as a preferred mode of use, further objects and advantages thereof, will best be understood by reference to the following detailed description of an illustrative embodiment when read in conjunction with the accompanying drawings, wherein:

FIG. 1 is a perspective view of the upper surface of the electro-optical sensor of the present invention;

FIG. 2 is a perspective view of the lower surface of the electro-optical sensor of the present invention;

FIG. 3 is a sectional view of the electro-optical sensor of the present invention;

FIG. 4 is an enlarged partial sectional view of the electro-optical sensor of the present invention;

FIG. 5 is a perspective view of the electro-optical sensor of the present invention wrapped around a patient's finger; and

FIG. 6 is a perspective view of the electro-optical sensor of the present invention wrapped around an infant's foot.

### DETAILED DESCRIPTION OF THE PREFERRED EMBODIMENT

With reference now to the figures and, in particular with reference to FIG. 1, there is depicted a perspective view of the upper surface of the electro-optical sensor of the electro-optical sensor 10 of the present invention which may be utilized for trans-illumination of a blood perfused portion of a human fingertip, toe, hand or infant's foot in order to measure light extinction during trans-illumination.

As illustrated, electro-optical sensor 10 is constructed utilizing a flexible initially substantially planar web-like support structure 12 which has an adhesive layer (not shown) on an upper surface thereof.

A compressible foam layer 14 is placed on the adhesive surface of web-like support structure 12. Compressible foam layer 14 is preferably constructed of a layer of polyether polyurethane foam between one-eighth inch and three-eighth inches in thickness and having a densibility of between one and three points pounds per cubic foot.

Present within compressible foam layer 14 are first aperture 16 and second aperture 18 which surround and overlie a light source and a photo-sensor in a manner which will be explained in greater detail herein.

A hook fabric tab 20 is attached to one end of sensor 10, and, in a manner which will be explained in greater detail herein, serves to mate with a loop fabric backing layer so that electro-optical sensor 10 maybe wrapped around a human fingertip, toe, hand or infant's foot. Finally, a cable 22 is utilized to connect the light source and photo-sensor to a meter capable of displaying and/or recording the oxygen saturation level in blood perfused tissue within the patient.

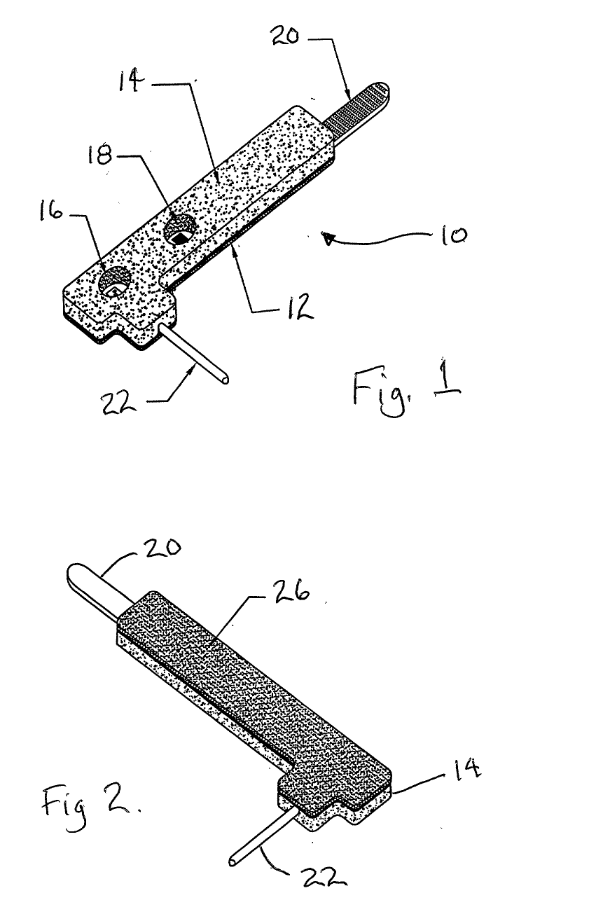

Referring now to FIG. 2, there is depicted a perspective sectional view of the lower surface of the electro-optical sensor 10 of the present invention. As illustrated, the lower surface of the initially substantially planar web-like support structure is covered by a loop fabric backing which, in combination with loop fabric backing 26 which, in combination with hook fabric tab 20 may be utilized to wrap electro-optical sensor 10 around a patient in a manner which will be illustrated in greater detail herein.

Referring now to FIG. 3, there is depicted a sectional view of electro-optical sensor 10 of the present invention. As illustrated, loop fabric backing 26 underlies compressible foam layer 14. Mounted to an adhesive layer (not shown) on the upper surface of loop fabric backing 26 are light source 30 and photo-sensor 32. As depicted, light source 30 is mounted having a light emitting surface facing away from the web-like support structure. Similarly, a photo-sensor 32 is mounted to the adhesive layer (not shown) on the upper surface of loop fabric backing 26. The photo-sensitive surface of the photo-sensor faces away from web-like support structure 12 in a manner similar to the description of light source 30.

As further illustrated in FIG. 3, it may be seen that first aperture 16 overlies light source 30. Similarly, second aperture 18 overlies photo-sensor 32. Thus, light source 30 and photo-sensor 32 are embedded within compressible foam layer 14 and will not, in typical application, contact the flesh of a patient.

Referring now to FIG. 4, there is depicted an enlarged partially sectional view of electro-optical sensor 10 of the present invention which depicts in greater detail the mounting of light source 30 and photo-sensor 32. As illustrated, web-like support structure 12 with its adhesive layer on the upper surface thereof serves to mount light source 30 and photo-sensor 32. Next, mounted over light source 30 and photo-sensor 32 are clear film patches 36 and 38. Clear film patches serve to further secure light source 30 and photo-sensor 32 to the adhesive layer which forms the upper surface of web-like support structure 12. As illustrated, film layer 14 is then placed onto the adhesive layer which forms the upper surface of web-like support structure 12 with first aperture 36 overlying light source 30 and second aperture 38 overlying photo-sensor 32.

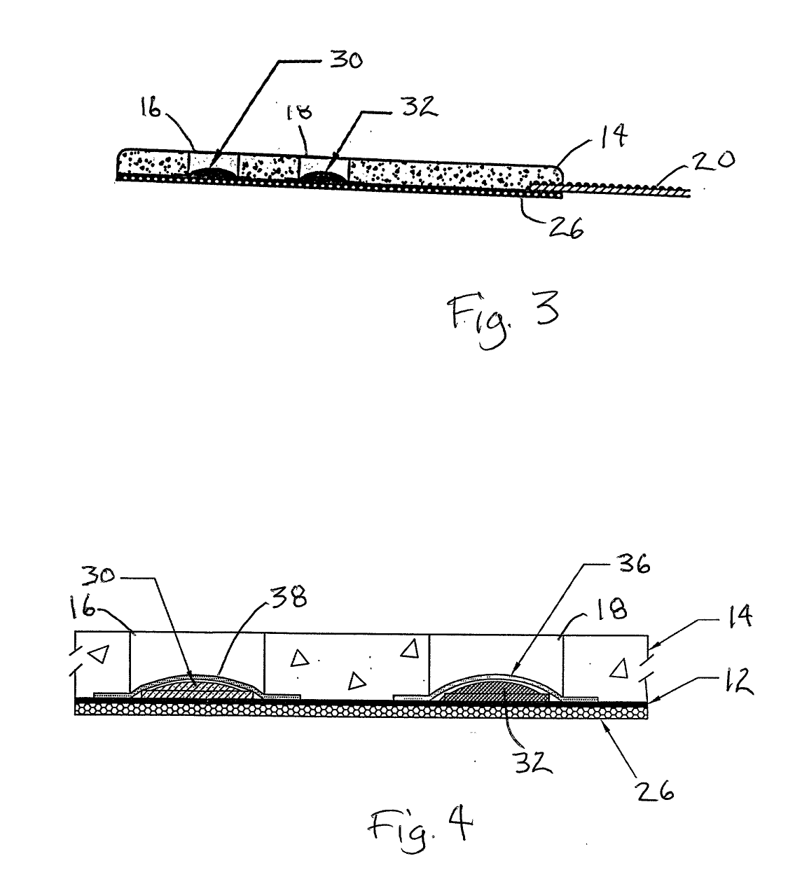

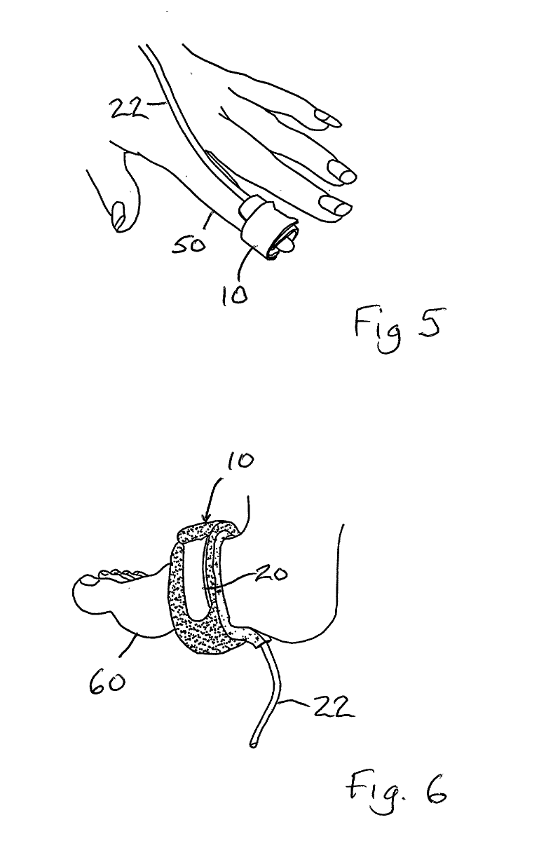

With reference now to FIG. 5, there is depicted a perspective view of the electro-optical sensor 10 of the present invention wrapped around a patient's finger 50. As illustrated, electro-optical sensor 10 may be easily wrapped around any human fingertip, toe, hand or infant's foot and, in the model depicted within FIG. 5, the spacing of light source 30 and photo-sensor 32 are such that when wrapped around an average human finger, toe, hand or infant's foot, light source 30 and photo-sensor 32 are placed facing each other on opposite sides of finger 50. In this manner, the blood perfused tissue within fingertip 50 may be trans-illuminated and the light extinction during trans-illumination may be measured.

Finally, referring to FIG. 6, there is depicted a perspective view of electro-optical sensor 10 of the present invention wrapped around an infant's foot 60. As illustrated, hook fabric tab 20 may be utilized to wrap electro-optical sensor 10 around patient's foot 60. In this depicted embodiment, the spacing of light source 30 and photo-sensor 32 is designed so that light source 30 and photo-sensor 32 are disposed opposite each other when electro-optical sensor is wrapped around the average size foot of a neo-natal infant.

As depicted herein, the present invention provides a non-adhesive flexible electro-optical sensor which may be repeatedly wrapped around a patient's appendage, removed and rewrapped without irritating the skin from adhesive chemicals or traumatizing the skin from removal of the sensor. The hook and loop closure provided make it simple and easy to wrap and unwrap the sensor, promoting any necessary inspection of the tissue at the monitoring site. The sensor elements are embedded within the compressible foam layer, insulating those elements from shock and ambient light.

The embodiments and examples set forth herein are presented in order to best explain the present invention and its practical application and, thereby, to enable those skilled in the art to make and use the invention. However, those skilled in the art will recognize that the foregoing description and examples have been presented for the purposes of illustration and example only. The description as set forth is not intended to be exhaustive or to limit the invention to the precise form disclosed. Many modifications and variations are possible in light of the above teaching without departing from the spirit and scope of the following claims.

What is claimed is:

1. A non-invasive electro-optical sensor for removable attachment to a patient for use in measuring light extinction during trans-illumination of the blood perfused tissue within the patient, said sensor comprising:
a flexible, initially substantially planar web-like support structure having an adhesive layer on an upper surface thereof and a loop fabric backing on a lower surface thereof;
a light source mounted on said adhesive layer of said substantially planar web-like support structure, said light source having a light emitting surface facing away from said web-like support structure;
a photo-sensor mounted on said adhesive layer of said substantially planar web-like support structure, said photo-sensor having a light responsive surface facing away from said web-like support structure;
a compressible foam layer mounted on said adhesive layer of said flexible, initially substantially planar web-like support structure, said compressible foam layer having a first aperture overlying said light source and a second aperture overlying said photo-sensor; and
a hook fabric tab attached at one end of said flexible, initially substantially planar web-like support structure.

2. The non-invasive electro-optical sensor according to claim 1, further including a clear film patch interposed between said light source and said compressible foam layer.

3. The non-invasive electro-optical sensor according to claim 1, further including a clear film patch interposed between said photo-sensor and said compressible foam layer.

4. The non-invasive electro-optical sensor according to claim 1, wherein said compressible foam layer is between one-eighth and three-eighth inches in thickness.

5. The non-invasive electro-optical sensor according to claim 1, wherein said compressible foam layer has a densibility of between one and three pounds per cubic foot.

6. The non-invasive electro-optical sensor according to claim 1, wherein said compressible foam layer is constructed of polyether polyurethane.

7. The non-invasive electro-optical sensor according to claim 1, wherein said light source and said photo-sensor are disposed a sufficient distance apart such that said light source and said photo-sensor are disposed opposite each other when said web-like support structure is wrapped around a patient's finger.

8. The non-invasive electro-optical sensor according to claim 1, wherein said light source and said photo-sensor are disposed a sufficient distance apart such that said light source and said photo-sensor are disposed opposite each other when said web-like support structure is wrapped around an infant's foot.
